(19) United States
(12) Patent Application Publication
(10) Pub. No.: US 2023/0172916 A1
(43) Pub. Date: Jun. 8, 2023

(54) COMPOSITION COMPRISING AT LEAST ONE PROTOBERBERINE ALKALOID AND METHOD FOR MANUFACTURING SAME

(71) Applicant: ELEONOR, Waterloo (BE)

(72) Inventors: Cristina LOIRA-PASTORIZA, Bruxelles (BE); Fabian PRIEM, Waterloo (BE)

(21) Appl. No.: 17/918,866

(22) PCT Filed: Apr. 16, 2021
(86) PCT No.: PCT/EP2021/059981

§ 371 Date: Oct. 13, 2022

(30) Foreign Application Priority Data
Apr. 17, 2020 (BE) 2020/5255

(51) Int. Cl. A61K 31/4375; A61K 9/14

(52) U.S. Cl. CPC A61K 31/4375; A61K 9/146

(57) ABSTRACT

A composition in the form of a thermoformed extrudate includes at least one protoberberine alkaloid and at least one polymer of natural and/or synthetic origin chosen from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, and derivatives and mixtures thereof.

The present invention relates to a composition comprising at least one protoberberine alkaloid, manufacturing method thereof and use thereof.

Within the meaning of the present invention, the term “protoberberine alkaloid” designates both protoberberine alkaloids of natural and/or vegetable origin and synthetic protoberberine alkaloids, but also all derivatives of protoberberine alkaloids such as, for example, protoberberine alkaloid salts. By way of example, the term “berberine” which designates a protoberberine alkaloid encompasses the derivatives of the berberine such as berberine salts, for example berberine hydrochloride or berberine sulphate.

Protoberberine alkaloids are nitrogenous, heterocyclic basic organic substances essentially of vegetable origin, having well-known and widely exploited therapeutic properties.

Protoberberine alkaloids are part of the family of isoquinolines. According to the publication by Da-Cunha et al. (The Alkaloids: Chemistry and Biology. Protoberberine Alkaloids. Elsevier, 2005, vol. 62, pages 1 to 75), the protoberberine alkaloids can be divided into ten types corresponding to the chemical structures as set out in table 1 below.

Among the type III protoberberine alkaloids is berberine having the chemical structure below and of particular interest.

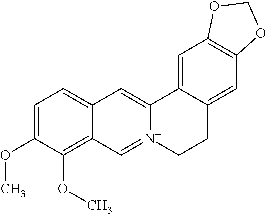 [SMILES: COc1ccc2cc3[n+](cc2c1OC)CCc1cc2c(cc1-3)OCO2]

TABLE 1
the different types of protoberberine alkaloids

| | |
|---|---|
| 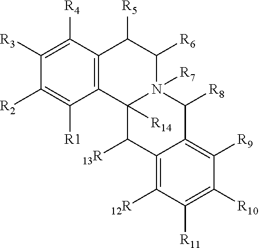 | Type I |
| 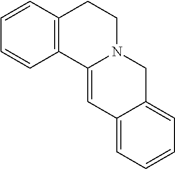 [SMILES: C1=C2c3ccccc3CCN2Cc2ccccc21] | Type II |
| 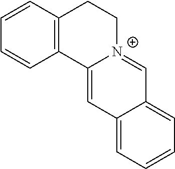 [SMILES: c1ccc2c(c1)CC[n+]1cc3ccccc3cc1-2] | Type III |
| 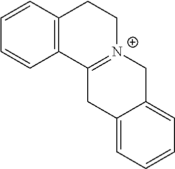 [SMILES: c1ccc2c(c1)CC1=[N+](CCc3ccccc31)C2] | Type IV |
| 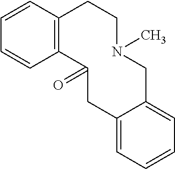 [SMILES: CN1CCc2ccccc2C(=O)Cc2ccccc2C1] | Type V |
| 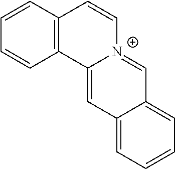 [SMILES: c1ccc2c[n+]3ccc4ccccc4c3cc2c1] | Type VI |
| 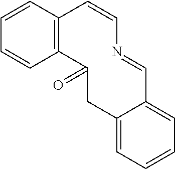 [SMILES: O=C1Cc2ccccc2/C=N/C=C\c2ccccc21] | Type VII |
| 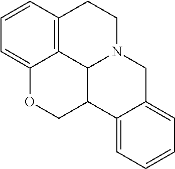 [SMILES: c1ccc2c(c1)CN1CCc3cccc4c3C1C2CO4] | Type VIII |
| 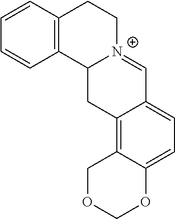 [SMILES: C1=[N+]2CCc3ccccc3C2Cc2c1ccc1c2COCO1] | Type IX |
| 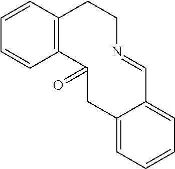 [SMILES: O=C1Cc2ccccc2/C=N/CCc2ccccc21] | Type X |

Hierarchically, berberine or 2,3-methylenedioxy-9,10-dimethoxy-protoberberine can therefore be classified as follows: alkaloids>isoquinolines>protoberberine alkaloids>berberine. It is a protoberberine alkaloid with multiple therapeutic potentials and therefore belongs to the group of isoquinolines, which are natural compounds present in the form of salts in certain plant species.

The vast majority of protoberberine alkaloids, including berberine, are heat-sensitive molecules, both lipophobic and hydrophobic, which is indeed reflected by their low solubility/miscibility in water but also in lipids and lipid derivatives.

It is worth noting that scientific studies report that berberine has a very low bioavailability of less than 5%, which is even only about 0.68% in laboratory rats subjected to experiments. In addition, efflux pumps, such as P-glycoprotein (P-gp) of the intestine and liver, have been shown to decrease the bioavailability of this alkaloid. P-gp is a membrane transporter responsible for the efflux of molecules absorbed by cells to the intestinal lumen. In the case of berberine, the majority of the absorbed molecule is rapidly expelled to the intestinal lumen by this glycoprotein.

It is widely recognised that the protoberberine alkaloids are compounds that are very slightly or completely insoluble in water in which they disperse only weakly or not at all, these compounds therefore have very low bioavailabilities. However, despite their low bioavailability/bioaccessibility (i.e. despite the small fraction of the administered dose which actually reaches the bloodstream in unchanged form) but also despite their low solubility and/or despite their low dispersion, in particular in the intestinal environment, the positive effects of protoberberine alkaloids on various pathologies make them molecules of interest for administration to humans and/or for veterinary use.

Therefore, many methodologies and methods have been developed in order to formulate these compounds in the form of spherical particles, flakes, pellets or even granules. In particular, extrusion techniques can be used such as, for example, the extrusion-spheronisation technique or the extrusion granulation technique (TSG or Tween Screw Granulation).

The extrusion-spheronisation technique allows the production of round particles with homogeneous size from a wet mass (containing an active substance and at least one excipient) which is passed through a grid having a predetermined mesh size before drying the particles thus obtained. More particularly, this method of powder shaping is based on the following steps of: mixing an active substance and at least one excipient, wet granulation (compaction) of the previously obtained mixture, extrusion of the compacted mixture to obtain an extrudate, spheronisation of the extrudate to form spherical particles/granules and drying of the spherical particles/granules obtained.

The extrusion granulation technique provides intermediate products for the preparation of tablets and capsules. This technique is based on a granulation (compaction) of substances in the form of powders in an extruder in order to form granules at the outlet thereof.

Unfortunately, it appears that the current formulations comprising protoberberine alkaloids are not very suitable because of their very low solubility or even their non-solubility and/or because of their very low dispersion or even their non-dispersion in aqueous phase and/or because of the weak release of these compounds from these formulations, which ultimately results in low bioavailability/bioaccessibility of these molecules of interest. It should be noted that it also appears that the current methods of manufacturing these formulations are restrictive and difficult to implement.

By the terms “dispersion in aqueous phase (in aqueous medium)”, it is understood, within the meaning of the present invention, a system composed of two phases in which one of the two phases, called dispersed phase, is finely divided in the other, called dispersing phase. This dispersion can be molecular (solution), colloidal (dispersion of submicron particles) or coarser (dispersion of particles greater than 1 μm). More particularly, according to the invention, the terms “dispersion in aqueous phase” designate suspensions, consisting of a solid phase dispersed in an aqueous (liquid) phase.

Within the meaning of the present invention, the term “solubility” designates the capacity of a substance, called solute, to dissolve in another substance, called solvent, to form a homogeneous mixture called solution.

The object of the invention is to overcome the drawbacks of the state of the art by providing (1) a composition comprising at least one protoberberine alkaloid, the solubility and/or dispersion of which in aqueous phase (aqueous medium) is increased such that the bioavailability of these compounds is significantly increased and (2) a method of manufacturing such a composition which is easy to implement, flexible, cost-effective and which ensures that said at least one protoberberine alkaloid is present and homogeneously spread in the final composition obtained.

Furthermore, the invention intends to provide a composition which is stable over time, i.e. which retains its properties in terms of solubility and/or dispersion of said at least one protoberberine alkaloid and which retains its properties in terms of release rate of this compound over time from a composition (formulation) according to the invention, particularly in aqueous phase and more particularly in the intestinal medium.

To at least partially resolve these problems, there is provided according to the invention, a composition in the form of a thermoformed extrudate comprising at least one protoberberine alkaloid and at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

In particular, according to the invention, said at least one polymer is a thermoplastic polymer, i.e. a polymer having the property of softening when it is sufficiently heated, but which, on cooling, becomes hard again.

According to one embodiment, there is therefore provided according to the invention, a composition in the form of a thermoformed extrudate comprising at least one protoberberine alkaloid and at least one protein of natural and/or synthetic origin as polymer.

By way of example, among the proteins, mention may be made of glycoproteins, collagens, collagen hydrolysates and/or collagen peptides and/or collagen polypeptides, pea proteins, wheat proteins, pumpkin proteins, nut proteins, rice proteins, derivatives thereof and mixtures thereof.

In particular and advantageously according to the invention, said collagen hydrolysates and/or said collagen peptides and/or collagen polypeptides are collagen hydrolysates and/or collagen peptides and/or collagen polypeptides unable/not having the capacity to gel, i.e. collagen hydrolysates and/or collagen peptides and/or non-gelling collagen polypeptides.

According to one embodiment, there is therefore provided according to the invention, a composition in the form of a thermoformed extrudate comprising at least one protoberberine alkaloid and at least one amino acid and/or at least one peptide and/or at least one polypeptide of natural and/or synthetic origin as polymer.

By way of example, among the amino acids, mention may be made of lysine, proline, tyrosine, arginine, derivatives thereof and mixtures thereof.

By way of example, among the peptides and the polypeptides, mention may be made of those resulting from the hydrolysis of collagen and derivatives thereof. The terms “collagen peptides” designate small, ordered groups of amino acids resulting from the fragmentation of the collagen molecule. Depending on the hydrolysis technique used, these groups are more or less important and more or less active.

The term “protein” designates a macromolecule consisting of different amino acids that are linked together. Protein is the most common element and the basic molecular unit of all living beings. Proteins are characterised by the length of their molecular chain.

The term “amino acid” designates a carboxylic acid which also has an amine functional group and the term “peptide” designates a polymer of amino acids.

The term “collagen” designates a protein composed of three associated alpha polypeptide chains. These chains are linked by hydrogen bonds between hydroxylysine and hydroxyproline and covalent bonds.

The terms “hydrolysed collagen” indicate that the collagen has been hydrolysed, in particular to make it more assimilable.

According to one embodiment, there is therefore provided according to the invention, a composition in the form of a thermoformed extrudate comprising at least one protoberberine alkaloid and at least one lipid of natural and/or synthetic origin as polymer.

By way of example, among the lipids of natural and/or synthetic origin, mention may be made of animal or vegetable waxes, microcrystalline waxes, animal or vegetable oils, hydrogenated animal or vegetable oils, phospholipids (lecithin, . . . ), ceramides, fatty acid esters, derivatives thereof and mixtures thereof.

According to one embodiment, there is therefore provided according to the invention, a composition in the form of a thermoformed extrudate comprising at least one protoberberine alkaloid and at least one dextrin of natural and/or synthetic origin as polymer.

By way of example, among the dextrins of natural and/or synthetic origin, mention may be made of pea dextrins and potato dextrins.

According to one embodiment, there is also provided according to the invention, a composition in the form of a thermoformed extrudate comprising at least one protoberberine alkaloid and at least one alginate of natural and/or synthetic origin as polymer.

By way of example, among the alginates, mention may be made of alginic acid, sodium alginate, calcium alginate, derivatives thereof and mixtures thereof.

According to one embodiment, there is also provided according to the invention, a composition in the form of a thermoformed extrudate comprising at least one protoberberine alkaloid and at least one oligosaccharide of natural and/or synthetic origin as polymer.

By way of example, among the oligosaccharides, mention may be made of raffinose, rhamminose, rhamnose, stachyose, verbascose, trehalose, lactose, lactulose, maltose, derivatives thereof and mixtures thereof.

According to one embodiment, there is also provided according to the invention, a composition in the form of a thermoformed extrudate comprising at least one protoberberine alkaloid and at least one cyclodextrin of natural and/or synthetic origin as polymer.

By way of example, among the cyclodextrins, mention may be made of α-cyclodextrin, β-cyclodextrin, γ-cyclodextrin, derivatives thereof and mixtures thereof.

According to one embodiment, there is also provided according to the invention, a composition in the form of a thermoformed extrudate comprising at least one protoberberine alkaloid and at least one hyaluronate of natural and/or synthetic origin as polymer.

By way of example, among the hyaluronates, mention may be made of hyaluronic acid, sodium hyaluronate, derivatives thereof and mixtures thereof.

According to one embodiment, there is also provided according to the invention, a composition in the form of a thermoformed extrudate comprising at least one protoberberine alkaloid and at least one carrageenan of natural and/or synthetic origin as polymer.

By way of example, among the carrageenans, mention may be made of k-carrageenan, i-carrageenan, λ-carrageenan, derivatives thereof and mixtures thereof.

By the term “extrudate”, it is understood, within the meaning of the present invention, a material which comes out of an extruder, in particular the die of an extruder.

By the terms “thermoformed extrudate”, it is understood, within the meaning of the present invention, a material which comes out of an apparatus, in particular which comes out of an extruder, in which it has undergone a transformation by the effect of heat, eventually by the combined effect of heat and shear forces of a worm. Such transformation by the effect of heat, eventually by the combined effect of heat and shear forces of a worm, can be obtained with the hot extrusion technique (HME or Hot Melt Extrusion).

In particular, a thermoformed extrudate according to the invention is an extrudate in which the active ingredient (said at least one protoberberine alkaloid) and/or said at least one polymer is/are melted.

In particular, the composition according to the invention, more particularly the composition in the form of a thermoformed extrudate according to the invention, is a solid dispersion in which said at least one protoberberine alkaloid is dispersed in said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

According to one embodiment, there is therefore provided according to the invention, a composition in the form of a thermoformed extrudate comprising at least one protoberberine alkaloid and at least one protein of natural and/or synthetic origin as polymer, said composition being a solid dispersion in which said at least one protoberberine alkaloid is dispersed in said at least one protein of natural and/or synthetic origin as polymer, which is/was melted.

According to one embodiment, there is therefore provided according to the invention, a composition in the form of a thermoformed extrudate comprising at least one protoberberine alkaloid and at least one amino acid and/or at least one peptide and/or at least one polypeptide of natural and/or synthetic origin as a polymer, said composition being a solid dispersion in which said at least one protoberberine alkaloid is dispersed in said at least one amino acid and/or in said at least one peptide and/or in said at least one polypeptide as polymer, which is/was melted.

According to one embodiment, there is therefore provided according to the invention, a composition in the form of a thermoformed extrudate comprising at least one protoberberine alkaloid and at least one dextrin of natural and/or synthetic origin as polymer, said composition being a solid dispersion in which said at least one protoberberine alkaloid is dispersed in said at least one dextrin of natural and/or synthetic origin as polymer, which is/was melted.

According to one embodiment, there is therefore provided according to the invention, a composition in the form of a thermoformed extrudate comprising at least one protoberberine alkaloid and at least one alginate of natural and/or synthetic origin as polymer, said composition being a solid dispersion in which said at least one protoberberine alkaloid is dispersed in said at least one alginate of natural and/or synthetic origin as polymer, which is/was melted.

According to one embodiment, there is therefore provided according to the invention, a composition in the form of a thermoformed extrudate comprising at least one protoberberine alkaloid and at least one oligosaccharide of natural and/or synthetic origin as polymer, said composition being a solid dispersion in which said at least one protoberberine alkaloid is dispersed in said at least one oligosaccharide of natural and/or synthetic origin as polymer, which is/was melted.

According to one embodiment, there is therefore provided according to the invention, a composition in the form of a thermoformed extrudate comprising at least one protoberberine alkaloid and at least one cyclodextrin of natural and/or synthetic origin as polymer, said composition being a solid dispersion in which said at least one protoberberine alkaloid is dispersed in said at least one cyclodextrin of natural and/or synthetic origin as polymer, which is/was melted.

According to one embodiment, there is therefore provided according to the invention, a composition in the form of a thermoformed extrudate comprising at least one protoberberine alkaloid and at least one hyaluronate of natural and/or synthetic origin as polymer, said composition being a solid dispersion in which said at least one protoberberine alkaloid is dispersed in said at least one hyaluronate of natural and/or synthetic origin as polymer, which is/was melted.

According to one embodiment, there is therefore provided according to the invention, a composition in the form of a thermoformed extrudate comprising at least one protoberberine alkaloid and at least one carrageenan of natural and/or synthetic origin as polymer, said composition being a solid dispersion in which said at least one protoberberine alkaloid is dispersed in said at least one carrageenan of natural and/or synthetic origin as polymer, which is/was melted.

Preferably, according to the invention, said thermoformed extrudate consists of a solid dispersion.

Advantageously, according to the invention, said solid dispersion consists of a glassy structure comprising a molecular mixture of said at least one protoberberine alkaloid and said at least one polymer.

In particular, a thermoformed extrudate according to the invention is an extrudate in which said at least one protoberberine alkaloid and/or said at least one polymer was/were melted to give rise to a solid dispersion consisting of a glassy structure comprising a molecular mixture of said at least one protoberberine alkaloid and said at least one polymer such that said at least one protoberberine alkaloid is dispersed within said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

By the terms “glassy structure”, it is understood, within the meaning of the present invention, a structure comprising/being formed by a molecular mixture of at least two compounds/molecules, at least one of these two compounds/molecules being at least partially in a non-crystalline form, in particular being at least partially in amorphous form.

By the terms “solid dispersion”, it is understood, within the meaning of the present invention, a mixture/system of at least two compounds/molecules, at least one of these two compounds/molecules being at least partially in a non-crystalline form, in particular being at least partially in amorphous form.

In particular, a “solid dispersion” consists of a molecular dispersion of an active ingredient/active substance at least partially in amorphous form within a polymeric matrix.

In particular, the composition according to the invention, more particularly the composition in the form of a thermoformed extrudate according to the invention, is a solid dispersion, in particular a solid dispersion consisting of a glassy structure, in which said at least one protoberberine alkaloid is dispersed in said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

A thermoformed extrudate according to the invention is obtained by hot thermoforming, in particular by hot thermoforming using the hot extrusion technique. According to the invention, hot thermoforming therefore relates more particularly to the hot extrusion technique.

There is therefore provided according to the invention a composition obtained by hot thermoforming in the form of a thermoformed extrudate obtained by hot thermoforming, in particular obtained by hot extrusion, said composition comprising at least one protoberberine alkaloid and at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

A thermoformed extrudate according to the invention can therefore be obtained using the hot extrusion technique which allows to carry out the molecular dispersion of an active ingredient (active substance) within a polymer matrix (within a polymer) to form solid dispersions. This solid dispersion is possible due to heat supply and eventually due to the stress applied by the movement of the worms on the material in an extruder. Finally, the hot extrusion gives rise, at the outlet, to the formation of a thermoformed extrudate in the form of a rope which, in particular, can then be pelletized or ground.

If the extrusion-spheronisation technique and the extrusion granulation technique as described above are carried out without heat supply (heating) and they typically require a liquid phase (generally water) to obtain spherical particles and/or granules, the hot extrusion technique can be carried out without supplying this liquid phase but relies on a heat supply (heating) to ensure a transformation of the material by thermoforming, i.e. a modification of the structure of at least one of the compounds/molecules used, in particular an at least partial transformation from the crystalline state to the amorphous state.

Moreover, if the extrusion-spheronisation technique and the extrusion granulation technique consist of an agglomeration of powder granules while trying as much as possible to preserve the initial properties of the constituents of these powders, the hot extrusion technique, on the contrary, leads to a transformation of the material, in particular to a “glassy structure” obtained by the action of heat (heating), the particles constituting the powders not all being present in their initial (native) crystalline form at the end of the hot extrusion method but having undergone a transformation by thermoforming.

According to the invention, preferably, said at least one protoberberine alkaloid comprises at least a first amorphous phase and eventually a second crystalline phase.

By the terms “comprises at least a first amorphous phase and eventually a second crystalline phase”, it is understood, within the meaning of the present invention, that said at least one protoberberine alkaloid can either comprise 100% by mass of an amorphous phase, or that it can simultaneously comprise a first amorphous phase and a second crystalline phase, the sum of the mass percentages of the first and second phases being in this case equal to 100. In other words, the composition according to the invention can comprise said at least one protoberberine alkaloid (1) completely in amorphous form or (2) partly in amorphous form (phase) and partly in crystalline form (phase).

It should be noted that a phase is considered as amorphous when the atoms constituting it do not respect any order at medium and long distances, which distinguishes it from a so-called crystalline phase.

The composition according to the invention is therefore preferably in the form of a thermoformed extrudate in which said at least one protoberberine alkaloid (active ingredient) comprises at least a first amorphous phase and eventually a second crystalline phase, which phases(s) is/are dispersed within at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

It has been determined, in the context of the present invention, that such a composition in the form of a thermoformed extrudate comprising at least one protoberberine alkaloid as active ingredient and at least a polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof has a significantly greater solubility and/or dispersion in aqueous phase (aqueous medium) of said at least one protoberberine alkaloid. Furthermore, it has been shown that such a composition according to the present invention has a significantly increased bioavailability/bioaccessibility of said at least one protoberberine alkaloid compared to the bioavailability/bioaccessibility observed for the current compositions.

Furthermore, in the context of the present invention, for a composition according to the invention, it has been determined that the protoberberine alkaloid+polymer mixture (selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin) is fluid, which allows an apparatus to be easily and homogeneously (homogeneous supply rate) supplied to obtain a thermoformed extrudate, for example a hot extruder. This is particularly advantageous in terms of industrial manufacturing since other polymers such as hydroxypropyl cellulose (HPC) in mixture with a protoberberine alkaloid (e.g. berberine or tetrahydropalmatine) lead to a non-fluid mixture [protoberberine alkaloid+polymer] which does not make it possible to appropriately supply (easily and with a homogeneous supply rate) an apparatus to obtain a thermoformed extrudate, for example a hot extruder, the mixture [protoberberine alkaloid+polymer] may even be trapped in the apparatus, in particular in the mixture [protoberberine alkaloid+polymer] supply area of the apparatus.

In addition, in the context of the present invention, for a composition according to the invention, it has been determined that the thermoformed extrudate obtained, in particular the thermoformed extrudate obtained by hot extrusion (HME), is brittle, which makes it easy to grind with conventional equipment in order to directly obtain a fluid powder which does not require the addition of excipients. This is particularly advantageous in terms of industrial manufacturing since other polymers such as hydroxypropylcellulose (HPC) in mixture with a protoberberine alkaloid (e.g. berberine or tetrahydropalmatine) lead to soft and elastic thermoformed extrudates whose grinding or transformation into pellets is very difficult and requires in particular the use of more complex and costly equipment and techniques such as cryogenic grinding which can also lead to degradation/alteration of the thermoformed extrudates (the thermoformed extrudates must be frozen at temperatures of about −90° C.). In addition, the powder obtained by cryogenic grinding remains sticky, which systematically requires the addition of an excipient.

According to the invention, the active ingredient, i.e. said at least one proto-berberine alkaloid is homogeneously dispersed/spread/distributed within at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, the active ingredient and/or said at least one polymer being melted during the manufacturing method by thermoforming implemented according to the invention and described below.

In addition, a composition according to the invention can be stored for several months without its properties being altered. In particular, it has been demonstrated that a composition according to the invention retains its properties in terms of solubility and/or dispersion in aqueous phase (aqueous medium) of said at least one protoberberine alkaloid and in terms of release rate of this compound over time from a composition/formulation according to the invention, particularly in aqueous phase.

Advantageously, according to the invention, said thermoformed extrudate comprises a thermoformed mixture of at least one protoberberine alkaloid and at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

Alternatively, according to the invention, said thermoformed extrudate consists of a thermoformed mixture of at least one protoberberine alkaloid and at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

There is therefore provided according to the invention, a thermoformed extrudate obtained by hot thermoforming, obtained in particular by hot extrusion, said thermoformed extrudate comprising a thermoformed mixture of at least one protoberberine alkaloid and at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

By the terms “thermoformed mixture”, it is understood, within the meaning of the present invention, a mixture which comes out of an apparatus, in particular which comes out of an extruder, in which it has undergone a transformation by the effect of heat, eventually by the combined effect of heat and shear forces of a worm. Such transformation by the effect of heat, eventually by the combined effect of heat and shear forces of a worm, can be obtained with the hot extrusion technique (HME or Hot Melt Extrusion).

In particular, a thermoformed mixture according to the invention is a mixture in which the active ingredient (said at least one protoberberine alkaloid) and/or said at least one polymer is/are melted was/were melted to give rise to a solid dispersion consisting of a glassy structure comprising a molecular mixture of said at least one protoberberine alkaloid and said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and of mixtures thereof, such that said at least one protoberberine alkaloid is dispersed within said at least one polymer.

According to the invention, said at least one protoberberine alkaloid comprises a first amorphous phase.

Advantageously, according to the invention, said at least one protoberberine alkaloid comprises predominantly at least a first amorphous phase.

Preferably, according to the invention, said at least one protoberberine alkaloid comprises 51 to 100% by mass of an amorphous phase and 0 to 49% of a crystalline phase.

By the terms “predominantly at least a first amorphous phase”, it is therefore understood, within the meaning of the present invention, that said at least one protoberberine alkaloid comprises 50 to 100% by mass of an amorphous phase and 0 to 50% of a crystalline phase, more particularly that said at least one protoberberine alkaloid comprises 51 to 100% by mass of an amorphous phase and 0 to 49% of a crystalline phase.

Advantageously, according to the invention, said at least one protoberberine alkaloid is selected from the group consisting of alborine, anibacanine, anisocycline, artavenustine, berberastine, berberine, berberubine, berlambine, canadine, capaurimine, capaurine, caseadine, caseamine, cavidine, cerasodine, cerasonine, cheilanthifoline, clarkeanidine, columbamine, constrictosine, coptisine, coreximine, corybulbine, corycavamine, corycavine, corydalidzine, corydalmine, corymotine, corynoxidine, corypalmine, corysamine, corytenchine, coulteropine, cryptopine, cyclanoline, dauricoside, discretamine, fississaine, govanine, groenlancidine, gusanlung-A, gusanlung-B, gusanlung-D, hunnemanine, jatrorrhizine, lambertine, lienkonine, malacitanine, manibacanine, mecambridine, muramine, orientalidine, pallimamine, palmatine, pessoine, phellodendrine, prechilenine, protopine, schefferine, scoulerine, sinactine, spiduxine, spinosine, stephabinamine, stephabine, stepharanine, stepholidine, stylopine, tetrahydropalmatine, thaicanine, thaipetaline, thalictricavine, thalidastine, thalifendine, xilopinine, yuanamide, dihydroberberine, dehydrocorydalmine, palmatrubine, dehydrodiscretamine, dehydrocheilanthifoline, demethylenberberine, epiberberine, pseudocoptisine, pseudopalmatine, pseudojatrorrhizine, pseudoepiberberine, pseudocolumbamine, dehydrodiscretine, dehydrocoreximine, thalifaurine, corysamine, dehydrothalictrifoline, dehydrothalictricavine, dehydrocorydaline, dehydroapocavidine, lincagenine, dehydrocapaurimine, 13-methyl-pseudoepiberberine, mequinine, derivatives thereof, salts thereof and mixtures thereof.

Preferentially, according to the invention, said proteins of natural and/or synthetic origin are selected from the group consisting of glycoproteins, collagens, collagen hydrolysates and/or collagen peptides and/or collagen polypeptides, vegetable proteins, animal proteins, derivatives thereof and mixtures thereof.

Particularly and advantageously according to the invention, said collagen hydrolysates and/or said collagen peptides and/or said collagen polypeptides are collagen hydrolysates and/or collagen peptides and/or collagen polypeptides unable/not having the capacity to gel, i.e. collagen hydrolysates and/or collagen peptides and/or non-gelling collagen polypeptides.

By the terms “collagen hydrolysate”, it is understood, within the meaning of the present invention, hydrolysed collagens and/or collagen peptides and/or collagen polypeptides.

By way of example, when said at least one protein of natural and/or synthetic origin is collagen or a collagen hydrolysate or at least a collagen peptide or at least a collagen polypeptide, in particular a non-gelling collagen hydrolysate or at least a non-gelling collagen peptide or at least a non-gelling collagen polypeptide, it may be a collagen or a collagen hydrolysate or at least a collagen peptide or at least a collagen polypeptide of animal origin (fish, pork, beef, . . . ).

By way of example, when said at least one protein of natural and/or synthetic origin is a vegetable protein, it may be a pumpkin, rice, wheat, pea or nut protein. This list is not exhaustive.

Preferentially, according to the invention, said collagens, said collagen hydrolysates and/or said collagen peptides and/or said collagen polypeptides, in particular said collagen hydrolysates and/or said collagen peptides and/or said non-gelling collagen polypeptides agents, have a molecular weight of between 50 and 300,000 Da, preferably between 100 and 275,000 Da, preferably between 150 and 250,000 Da, preferably between 200 and 225,000 Da, preferably between 250 and 200,000 Da, preferably between 300 and 175,000 Da, preferably between 350 and 150,000 Da, preferably between 400 and 125,000 Da, preferably between 450 and 100,000 Da, preferably between 500 and 75,000 Da, preferably between 550 and 50,000 Da, preferably between 600 and 40,000 Da, preferably between 650 and 30,000 Da, preferably between 700 and 20,000 Da, preferably between 750 and 10,000 Da, preferably between 800 and 9,000 Da, preferably between 850 and 8,000 Da, preferably between 900 and 7,000 Da, preferably between 950 and 6,000 Da, preferably between 1,000 and 5,000 Da, preferably between 1,050 and 4,000 Da, preferably between 1,100 and 3,000 Da, preferably between 1,150 and 2,000 Da, preferably between 1,200 and 1,000 Da.

Advantageously, according to the invention, said collagens, said collagen hydrolysates and/or said collagen peptides and/or said collagen polypeptides, in particular said collagen hydrolysates and/or said collagen peptides and/or said non-gelling collagen polypeptides, have a molecular weight of between 1,000 and 300,000 Da, preferably between 1,500 and 150,000 Da, preferably between 2,000 and 60,000 Da.

Preferably, according to the invention, said collagens, said collagen hydrolysates and/or said collagen peptides and/or said collagen polypeptides, in particular said collagen hydrolysates and/or said collagen peptides and/or said non-gelling collagen polypeptides, have a molecular weight of 50 Da or 100 Da or 150 Da or 200 Da or 250 Da or 300 Da or 350 Da or 400 Da or 450 Da or 500 Da or 550 Da or 600 Da or 650 Da or 700 Da or 750 Da or 800 Da or 850 Da or 900 Da or 950 Da or 1,000 Da or 1,100 Da or 1,200 Da or 1,300 Da or 1,400 Da or 1500 Da or 1,600 Da or 1,700 Da or 1,800 Da or 1,900 Da or 2,000 Da or 2,500 Da or 3,000 Da or 3,500 Da or 4,000 Da or 4,500 Da or 5,000 Da or 5,500 Da or 6,000 Da or 6,500 Da or 7,000 Da or 7,500 Da or 8,000 Da or 8,500 Da or 9,000 Da or 9,500 Da or 10,000 Da or 12,500 Da or 15,000 Da or 17,500 Da or 20,000 Da or 22,500 Da or 25,000 Da or 27,500 Da or 30,000 Da or 32,500 Da or 35,000 Da or 37,500 Da or 40,000 Da or 42,500 Da or 45,000 Da or 47,500 Da or 50,000 Da or 55,000 Da or 60,000 Da or 65,000 Da or 70,000 Da or 75,000 Da or 80,000 Da or 85,000 Da or 90,000 Da or 100,000 Da or 110,000 Da or 120,000 Da or 130,000 Da or 140,000 Da or 150,000 Da or 160,000 Da or 170,000 Da or 180,000 Da or 190,000 Da or 200,000 Da or 210,000 Da or 220,000 Da or 230,000 Da or 240,000 Da or 250,000 Da or 260,000 Da or 270,000 Da or 280,000 Da or 290,000 Da or 300,000 Da.

According to one embodiment according to the invention, when said at least one protein of natural and/or synthetic origin is collagen, the latter has a molecular weight of between 900 and 7,000 Da, preferably a molecular weight of between 950 and 5,000 Da, preferentially a molecular weight of between 1,000 and 3,000 Da.

According to one embodiment according to the invention, when said at least one protein of natural and/or synthetic origin is collagen, the latter has a molecular weight of 2,000 Da or 3,000 Da or 5,000 Da or 50,000 Da.

According to one embodiment according to the invention, when said at least one protein of natural and/or synthetic origin is a hydrolysed collagen or a collagen peptide or a collagen polypeptide, in particular a non-gelling hydrolysed collagen or a non-gelling collagen peptide or a non-gelling collagen polypeptide, the latter has a molecular weight of between 900 and 6,000 Da, preferably a molecular weight of between 950 and 5,000 Da, preferentially a molecular weight of between 1,000 and 3,000 Da.

According to one embodiment according to the invention, when said at least one protein of natural and/or synthetic origin is a hydrolysed collagen or a collagen peptide or a collagen polypeptide, in particular a non-gelling hydrolysed collagen or a non-gelling collagen peptide or a non-gelling collagen polypeptide, the latter has a molecular weight of 2,000 Da or 3,000 Da or 5,000 Da or 50,000 Da.

According to one embodiment, the composition according to the invention further comprises at least one additional polymer of natural and/or synthetic origin, for example polysaccharide, polyvinyl acetate, polyvinylpyrrolidone (PVP), polyvinylpyrrolidone-co-vinyl acetate, polyethylene-co-vinyl acetate, polyvinyl acetate-co-methacrylic acid, polyethylene oxide, polylactide-co-glycolide, polyvinyl alcohol, polycarbophil, polycaprolactone, carnauba wax, ethylene-vinyl copolymer, lecithin, castor oil, hydrogenated soybean oil, waxes, isomalt, derivatives thereof and mixtures thereof.

Advantageously, the composition according to the invention further comprises at least one plasticizer. The addition of a plasticizer to a composition according to the invention allows for obtaining a composition according to the invention through a manufacturing method where temperatures below the melting points of said at least one protoberberine alkaloid and polymer can be used in order to ensure melting of these two compounds and the dispersion/spread/distribution of said at least one protoberberine alkaloid within the polymer.

Preferentially, according to the invention, said plasticizer is selected from the group consisting of polyols, lipids, lecithins, sucrose esters, water, triethyl citrate, polyethylene glycol, glycerol, dibutyl sebate, butyl stearate, glycerol monostearate, diethyl phthalate, derivatives thereof and mixtures thereof.

According to the invention, the preferred plasticizers are glycerol, water, polyethylene glycol and triethyl citrate.

Preferably, the composition according to the invention further comprises at least one additive selected from the group consisting of lubricating agents, surfactants, antioxidants, chelating agents, derivatives thereof and mixtures thereof.

By way of example, the following compounds can be used, alone or in a mixture, as lubricating agents in a composition according to the invention: glycerol dibehenate, talc, silica, stearic acid, boric acid, waxes, sodium oleate, sodium acetate, magnesium stearate, calcium stearate, sodium stearate, sodium benzoate, sodium lauryl sulphate, glycerol distearate, glycerol palmitostearate, microcrystalline cellulose or polyoxyl-8-glycerides.

By way of example, the following compounds can be used, alone or in a mixture, as surfactants in a composition according to the invention: Pluronic®, Span®, Cremophor®, polysorbates (Tween®, . . . ), vitamin E TPGS and sodium ducosate.

By way of example, the following compounds can be used, alone or in a mixture, as antioxidants and/or chelating agents in a composition according to the invention: butylated hydroxytoluene, butylated hydroxyanisole, EDTA, citric acid and vitamin E.

Advantageously, the composition according to the invention further comprises at least one polyphenolic additional compound selected from the group consisting of phenolic acids, stilbenes, phenolic alcohols, lignans, flavonoids, derivatives thereof and mixtures thereof. In particular, the glycosylated and aglycone forms of polyphenols are envisaged as an additional active ingredient according to the present invention. More particularly, within the meaning of the present invention, the term “polyphenol” designates both polyphenols of natural origin and synthetic polyphenols, but also all polyphenol derivatives.

By way of example, within the meaning of the present invention, as phenolic acids, mention may be made of derivatives of hydroxybenzoic acid (gallic acid, tannic acid, . . . ) and derivatives of hydroxycinamic acid (curcumin, coumaric acid, caffeic acid, ferulic acid, . . . ).

By way of example, within the meaning of the present invention, as stilbenes, mention may be made of resveratrol, sirtinol, piceatannol or polydatin.

By way of example, within the meaning of the present invention, as flavonoids mention may be made of flavanols (quercetin, myricetin, kaempferol, isorhamnetin, morin, rutin, tiliroside, trihydroxyethylrutin, fisetin, . . . ), flavones (apigenin, luteolin, baicalein, chrysin, diosmin, nobiletin, tangeretin, wogonin, aminogenistein, . . . ), flavanones (bavachine, 8-isopentenyl-naringenin, isoxanthohumole, naringenin, eriodictyole, hesperetin, silybin, taxifolin, . . . ), isoflavones (genistein, daidzein, daidzin, formonetin, genistin, neobavaisoflavone, pueranine, . . . ), antocianidins (cyanidin, pelargonidin, delphinidin, petunidin, malvidin, . . . ) and flavanols (cathechins, gallocatechin, epigallocatechin gallate,

According to the invention, said at least one additional polyphenolic active ingredient constitutes an inhibitor/modulator of efflux pumps, including P-gp.

Preferentially, the composition according to the invention further comprises at least an inhibitor and/or a modulator of the activity of P-gp.

Advantageously, the composition according to the invention further comprises at least a hydrotropic agent, i.e. an agent which does not form micelles and which is capable of solubilising insoluble molecules. Hydrotropic agents generally consist of two parts, a hydrophilic part and a hydrophobic part.

By way of example, as hydrotropic agent mention may be made of citric acid, benzoic acid, sodium salicylate, sodium benzoate, sodium acetate, sodium ascorbate, potassium citrate, urea, caffeine, nicotinamide, N,N-dimethylurea, N,N-diethylnicotinamide, N,N-dimethylbenzamide, resorcinol, pyrogallol, catechol, naphthol, sodium dodecylsulphate, procaine HCl, polyethylene glycol and mannitol. This list is not exhaustive and the hydrotropic agents mentioned in particular in the following publications are covered by the present invention: Majeed et al. Hydrotrophy Novel solubility enhancement technique: A review. Int J Pharm Self & Res 2019, 10(3): 1025-36; Arjaria et al. Hydrotropic solubilization. Int J Pharm Phytopharmacol Res 2013, 3(1):11-23.

Preferably, the composition according to the invention is packaged in the form of pellets, flakes, granules, powders, effervescent or non-effervescent tablets, injectable or non-injectable solutions, suspensions, gels, ointments or in any other suitable form approved for administration to an animal or to a human being. Other embodiments of a composition according to the invention are indicated in the appended claims.

An object of the invention is also to provide a manufacturing method, in particular a thermoforming manufacturing method, of a composition in the form of a thermoformed extrudate according to the invention, characterised in that it comprises the following steps:

a) a step of providing, in a simultaneous or delayed manner, at least one protoberberine alkaloid and at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, for feeding an apparatus, for example an extruder,

b) a step of mixing, in said apparatus, for example in an extruder, said at least one protoberberine alkaloid and said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, to form a mixture, and

c) a step of thermoforming, in particular a step of hot extrusion, of said mixture obtained in step b) in said apparatus, for example in an extruder, to obtain a thermoformed extrudate.

Such a method according to the invention gives rise to a composition in the form of a thermoformed extrudate, i.e. obtained by thermoforming and more particularly by hot extrusion, in which said at least one protoberberine alkaloid as active ingredient preferentially comprises at least a first amorphous phase and eventually a second crystalline phase dispersed within said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof. This composition according to the invention has a significantly greater solubility and/or dispersion in aqueous phase (aqueous medium) of said at least one protoberberine alkaloid and simultaneously a significantly increased bioavailability of said at least one protoberberine alkaloid compared to the solubilities and bioavailabilities of these compounds of the current compositions. It has been shown that the composition according to the invention is in the form of a thermoformed extrudate in which said at least one protoberberine alkaloid (active ingredient) preferentially comprising at least a first amorphous phase and eventually a second crystalline phase is dispersed within said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

It has also been shown, in the context of the present invention, that said at least one protoberberine alkaloid is not degraded even during the thermoforming manufacturing method of the composition in the form of a thermoformed extrudate which nevertheless involves subjecting said at least one protoberberine alkaloid to high temperature (HME). Furthermore, it has also been demonstrated that said at least one protoberberine alkaloid in a composition in the form of a thermoformed extrudate according to the invention is homogeneously spread (distributed) therein.

More particularly, the hot extrusion (Hot Melt Extrusion—HME) carried out according to the thermoforming manufacturing method according to the invention leads to melting of the active ingredient (said at least one protoberberine alkaloid) and/or said at least one polymer at a temperature greater than or equal to their melting point.

However, according to some embodiments of a composition according to the invention, melting of the active ingredient and polymer can occur at a temperature below their melting point. This is for example the case if the composition according to the invention comprises a plasticizer or if the active ingredient itself has plasticizing properties. Such a melting of the active ingredient and/or polymer leads to a solid dispersion in which the active ingredient (said at least one protoberberine alkaloid) preferentially comprising at least a first amorphous phase and eventually a second crystalline phase is homogeneously dispersed/spread/distributed within said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

Advantageously, the method according to the invention comprises a prior step of pre-mixing said at least one protoberberine alkaloid and said at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and of mixtures thereof so as to form a pre-mixture to be fed into the apparatus, for example an extruder.

Preferably, according to the method according to the invention, said thermoforming step, in particular said hot extrusion step, is carried out at an extrusion temperature or thermoforming temperature of between 20 and 180° C., preferably at a temperature of between 40 and 115° C., preferentially at a temperature of between 80 and 90° C., preferably at a temperature of 115° C., preferably at a temperature of 110° C., preferably at a temperature of 100° C.

Advantageously, according to the method according to the invention, said thermoforming step, in particular said hot extrusion step, is carried out at a rotation speed of an extrusion screw of between 20 and 900 rpm, preferably of between 50 and 300 rpm, preferably of between 100 and 250 rpm, preferentially of 250 rpm, preferentially of 100 rpm.

Preferentially, the method according to the invention comprises an additional cooling step at the outlet of the apparatus, for example at the outlet of an extruder.

Advantageously, the method according to the invention comprises an additional step of processing the thermoformed extrudate at the outlet of the apparatus, for example at the outlet of an extruder, for example cutting using a pelletiser and/or grinding of said thermoformed extrudate.

Other embodiments of the method according to the invention are indicated in the appended claims.

The present invention also relates to a composition in the form of a thermoformed extrudate obtained according to the method according to the invention, said composition comprising at least one protoberberine alkaloid as active ingredient and at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, said at least one protoberberine alkaloid preferentially comprising at least a first amorphous phase and eventually a second crystalline phase.

In other words, the present invention further relates to a composition in the form of a thermoformed extrudate obtained by hot extrusion (HME—Hot Melt Extrusion), said composition comprising at least one protoberberine alkaloid as active ingredient and at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, said at least one protoberberine alkaloid preferentially comprising at least a first amorphous phase and eventually a second crystalline phase.

The present invention further relates to a use of a composition according to the invention as food supplement and/or cosmetic product and/or medicine for human or veterinary use.

In particular, the present invention relates to a composition for use in the preventive and/or curative treatment, in humans and/or in animals, of pathologies related to the cardiovascular system (hypotension, vasoconstriction, ventricular hypertrophy, arrhythmia, . . . ), pathologies related to blood system (cholesterolemia, platelet aggregation, . . . ), pathologies related to the gastrointestinal system (diarrhoea, digestive inflammation, modulation of the intestinal microbiota, dyspepsia, gastroesophageal reflux, . . . ), pathologies related to the premature aging of cells, pathologies related to the endocrine system (hyperglycaemia, . . . ), pathologies related to the immune system, pathologies related to the central nervous system, skin diseases, diseases due to the presence of microorganisms and cancers (anti-tumoral, . . . ), and in the preventive and/or curative treatment of diabetes.

More particularly, the present invention relates to a composition for use in the preventive and/or curative treatment, in humans and/or in animals, of diseases related to premature aging of cells, obesity, diabetes, hypercholesterolemia, metabolic syndrome and irritable bowel syndrome (IBS), non-alcoholic hepatic steatosis (NAFLD+NASH or fatty liver disease).

Other forms of use of a composition according to the invention are indicated in the appended claims.

A composition according to the invention having preferentially anti-inflammatory, lipid-lowering, antioxidant, antithrombotic, antitumor, antidiabetic, hepatoprotective, nefroprotective, immunoregulatory, antipsychotic properties as well as neuroprotective properties.

Other characteristics, details and advantages of the invention will emerge from the examples given below, in a non-limiting way and with reference to the appended figures.

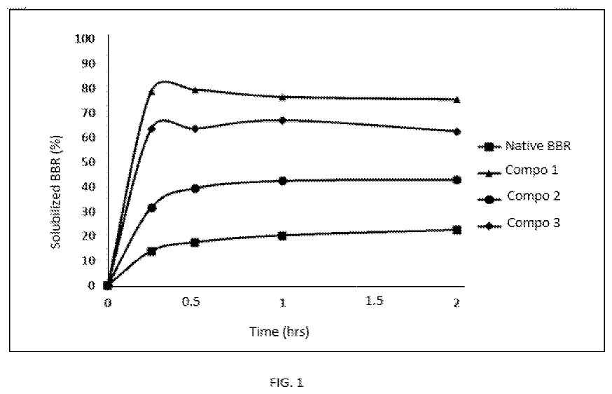

FIG. 1 is a graph illustrating the solubilisation rate of a protoberberine alkaloid, in this case the solubilisation rate of berberine over time, from examples of compositions according to the invention and from a composition not representing the object of the present invention.

### EXAMPLES

### Example 1: Thermoforming Manufacturing Method of Compositions According to the Invention in the Form of a Thermoformed Extrudate

Thermoformed compositions according to the invention comprising at least one protoberberine alkaloid, such as those of the Example 2 below, were obtained according to the following method further representing the object of the present invention:

a) a step of pre-mixing at least one protoberberine alkaloid in the crystalline state in powder form and at least one polymer selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof;

b) a step of providing said pre-mixture formed in step a) to feed an extruder of the Process 11 Hygienic type from Thermo Fisher Scientific®;

c) a step of mixing, in said extruder, said pre-mixture to obtain a mixture;

d) a step of thermoforming, by hot extrusion, said mixture obtained in step c) in said extruder to obtain a thermoformed extrudate, the hot extrusion step being carried out at a rotation speed of an extrusion screw of 100 rpm and at a temperature of between 20 and 80° C.;

e) a step of cooling, at the outlet of the extruder, said thermoformed extrudate obtained in step d); and

f) a step of cutting/grinding, using a grinder, the cooled thermoformed extrudate obtained in step e) so as to obtain a homogeneous powder.

The thermoforming temperature at which the hot extrusion step is carried out is determined by the type of constituents used, in particular according to the type of polymer and/or plasticizer used, which the skilled person is able to determine. Furthermore, a skilled person, in particular according to the type of extruder used and in accordance with the general principle of hot extrusion (HME), is able to define possible temperature steps in different areas along the extrusion screw(s) such that there is a gradual increase in temperature within the material transported by the extrusion screw(s), this in a feed direction of the material within the extruder. Typically, between areas defined along the extrusion screw(s), temperature differences of about 0 to 40° C. are observed. For example, in the context of the present invention, the compositions tested below were obtained in a Process 11 Hygienic type extruder from Thermo Fisher Scientific® having 8 temperature areas which are as follows in a feed direction of the material moving at a speed of 100 rpm: 20° C.-20° C.-20° C.-60° C.-80° C.-80° C.-80° C. and 80° C.

### Example 2: Solubility Tests of Thermoformed Compositions According to the Invention

Thermoformed compositions, obtained according to the manufacturing method described in example 1, were tested in terms of solubility of berberine. This solubility was measured over time from the thermoformed extrudates obtained according to the invention. As indicated above, the thermoformed extrudates are in the form of a homogeneous powder (ground material) in which berberine (a protoberberine alkaloid) preferentially comprises at least one amorphous phase.

The solubility tests were carried out with a paddle dissolution apparatus from about 1.5 g of each of the thermoformed extrudates, at a temperature of 37° C., with stirring at 50 rpm in 900 ml of a dissolution medium HCl 0.1 N. These solubility tests were carried out according to the recommendations of the pharmacopoeia Ph. Eur. 9.0 (Recommendations on Dissolution Testing). At determined times (after 15 min, after 30 min, after 1 h and after 2 h), a sample of 1 ml of mixture was taken to carry out a solubility test.

In order to carry out the solubility tests, each of the tested samples was filtered through a filter (PET, pore size: 0.45 Macherey Nagel) then diluted in the HPLC mobile phase before HPLC analysis (column: Nucleodur 100-5 EC C18 125/4 (Macherey-Nagel); mobile phase: 70%-A (Water-TFA 0.1%) and 30% B (ACN-TFA 0.1%); flow rate: 0.8 ml/min; loop: 20 μl, t°=40° C.).

The thermoformed compositions according to the invention (Compo 1 and Compo 3) given in Table 1 were formulated according to the method of the invention and tested in terms of solubility over time according to the principle indicated above. Berberine in native crystalline form and in powder form (native BBR) was used as control. A composition not representing the object of the invention (Compo 2) was tested for comparison with starch as polymer. The amounts mentioned in Table 1 are weight percentages of the compounds used (subjected to the method according to the invention) relative to the total weight of the composition.

TABLE 1

| | Berberine | Protein | Starch | Dextrin | Glycerol |
|---|---|---|---|---|---|
| | (1) | (2) | (4) | (5) | (3) |
| Compo 1 | 40 | 50 | 0 | 0 | 10 |
| Compo 2 | 40 | 0 | 50 | 0 | 10 |
| Compo 3 | 40 | 0 | 0 | 50 | 10 |

(1) Berberine HCl (Shanghai Freemen)
(2) Non-gelling collagen peptides Peptan ® B 5000 HD (with a molecular weight of 5,000 Da) (Ingrizo)
(3) Glycerol (Brenntag)
(4) Modified starch: Cleargum ® CB 90 (Roquette)
(5) Pea dextrin: Tackidex ® C760 (Roquette)

The results obtained are shown in FIG. 1. The results obtained are shown in FIG. 1. As can be seen, the solubilities measured for berberine from compositions in the form of thermoformed extrudates according to the invention (Compo 1—non-gelling collagen peptides as polymer; Compo 3—dextrin as polymer) are higher compared to the control (native BBR) and are also higher compared to a composition not representing the object of the present invention and comprising starch as polymer (Compo 2).

The present invention has been described in relation to specific embodiments, which are purely illustrative and should not be construed as limiting. In general, it is obvious to a skilled person that the present invention is not limited to the examples illustrated and/or described above.

The use of the verbs “to comprise”, “to include”, “to contain”, or any other variant, as well as their conjugations, cannot in any way exclude the presence of elements other than those mentioned.

The use of the indefinite article “a”, “an”, or the definite article “the”, to introduce an element does not exclude the presence of a plurality of these elements.

1. A composition in the form of a thermoformed extrudate comprising at least one protoberberine alkaloid and at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

2. The composition according to claim 1, wherein said thermoformed extrudate comprises a thermoformed mixture of at least one protoberberine alkaloid and at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.

3. The composition according to claim 1, wherein said at least one protoberberine alkaloid comprises predominantly at least a first amorphous phase.

4. The composition according to claim 3, wherein said at least one protoberberine alkaloid comprises 51 to 100% by mass of an amorphous phase and 0 to 49% of a crystalline phase.

5. The composition according to claim 1, wherein said at least one protoberberine alkaloid is selected from the group consisting of alborine, anibacanine, anisocycline, artavenustine, berberastine, berberine, berberubine, berlambine, canadine, capaurimine, capaurine, caseadine, caseamine, cavidine, cerasodine, cerasonine, cheilanthifoline, clarkeanidine, columbamine, constrictosine, coptisine, coreximine, corybulbine, corycavamine, corycavine, corydalidzine, corydalmine, corymotine, corynoxidine, corypalmine, corysamine, corytenchine, coulteropine, cryptopine, cyclanoline, dauricoside, discretamine, fississaine, govanine, groenlancidine, gusanlung-A, gusanlung-B, gusanlung-D, hunnemanine, jatrorrhizine, lambertine, lienkonine, malacitanine, manibacanine, mecambridine, muramine, orientalidine, pallimamine, palmatine, pessoine, phellodendrine, prechilenine, protopine, schefferine, scoulerine, sinactine, spiduxine, spinosine, stephabinamine, stephabine, stepharanine, stepholidine, stylopine, tetrahydropalmatine, thaicanine, thaipetaline, thalictricavine, thalidastine, thalifendine, xilopinine, yuanamide, dihydroberberine, dehydrocorydalmine, palmatrubine, dehydrodiscretamine, dehydrocheilanthifoline, demethylenberberine, epiberberine, pseudocoptisine, pseudopalmatine, pseudojatrorrhizine, pseudoepiberberine, pseudocolumbamine, dehydrodiscretine, dehydrocoreximine, thalifaurine, corysamine, dehydrothalictrifoline, dehydrothalictricavine, dehydrocorydaline, dehydroapocavidine, lincagenine, dehydrocapaurimine, 13-methyl-pseudoepiberberine, mequinine, derivatives thereof, salts thereof and mixtures thereof.

6. The composition according to claim 1, wherein said proteins of natural and/or synthetic origin are selected from the group consisting: of glycoproteins, collagens, non-gelling collagen hydrolysates, non-gelling collagen peptides, non-gelling collagen polypeptides, vegetable proteins, animal proteins, derivatives thereof and mixtures thereof.

7. The composition according to claim 6, wherein said collagens and/or said non-gelling collagen hydrolysates and/or said non-gelling collagen peptides and/or said non-gelling collagen polypeptides have a molecular weight of between 50 and 300,000 Da.

8. The composition according to claim 1, further comprising at least one plasticizer.

9. The composition according to claim 8, wherein said at least one plasticizer is selected from the group consisting of polyols, lipids, lecithins, sucrose esters, water, triethyl citrate, polyethylene glycol, glycerol, dibutyl sebate, butyl stearate, glycerol monostearate, diethyl phthalate, derivatives thereof and mixtures thereof.

10. The composition according to claim 1, further comprising at least one additive selected from the group consisting: of lubricating agents, surfactants, antioxidants, chelating agents, derivatives thereof and mixtures thereof.

11. The composition according to claim 1, further comprising at least one polyphenolic first additional compound selected from the group consisting of phenolic acids, stilbenes, phenolic alcohols, lignans, flavonoids, derivatives thereof and mixtures thereof.

12. The composition according to claim 1, which is packaged in the form of pellets, flakes, granules, powders, effervescent or non-effervescent tablets, injectable or non-injectable solutions, suspensions, gels, ointments or in a suitable form approved for administration to an animal or to a human being.

13. A method for manufacturing a composition in the form of a thermoformed extrudate according to claim 1, comprising the following steps:
a) providing, in a simultaneous or delayed manner, at least one protoberberine alkaloid and at least one polymer of natural and/or synthetic origin selected from the group consisting of: proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, for feeding an apparatus,
b) mixing, in said apparatus, said at least one protoberberine alkaloid and said at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof, to form a mixture, and
c) thermoforming of said mixture obtained in step b) in said apparatus, to obtain a thermoformed extrudate.

14. The method according to claim 13, comprising a prior step of pre-mixing said at least one protoberberine alkaloid and said at least one polymer of natural and/or synthetic origin selected from the group consisting of: proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and of mixtures thereof so as to form a pre-mixture to be fed into said apparatus.

15. The method according to claim 13, wherein said thermoforming is carried out at an extrusion temperature of between 20 and 180° C.

16. The method according to claim 13, wherein said thermoforming is carried out at a rotation speed of an extrusion screw of between 20 and 900 rpm.

17. The method according to claim 13, comprising an additional cooling step at the outlet of the apparatus.

18. The method according to claim 13, comprising an additional step of processing the thermoformed extrudate at the outlet of the apparatus.

19. The composition in the form of a thermoformed extrudate according to claim 1 for preventive and/or curative treatment, in humans and/or in animals, of pathologies related to the cardiovascular system (hypotension, vasoconstriction, ventricular hypertrophy, arrhythmia, . . . ), pathologies related to blood system (cholesterolemia, platelet aggregation, . . . ), pathologies related to the gastrointestinal system (diarrhoea, digestive inflammation, modulation of the intestinal microbiota, dyspepsia, gastroesophageal reflux, . . . ), pathologies related to the premature aging of cells, pathologies related to the endocrine system (hyperglycaemia, . . . ), pathologies related to the immune system, pathologies related to the central nervous system, skin diseases, diseases due to the presence of microorganisms and cancers (anti-tumoral, . . . ), in the preventive and/or curative treatment of diabetes, diseases related to premature aging of cells, obesity, hypercholesterolemia, metabolic syndrome and irritable bowel syndrome (IBS), non-alcoholic hepatic steatosis (NAFLD+NASH or fatty liver disease).

20. The composition in the form of a thermoformed extrudate obtained according to the method of claim 13, said composition comprising at least one protoberberine alkaloid as active ingredient and at least one polymer of natural and/or synthetic origin selected from the group consisting of proteins of natural and/or synthetic origin, amino acids, peptides and polypeptides of natural and/or synthetic origin, lipids of natural and/or synthetic origin, dextrins of natural and/or synthetic origin, alginates of natural and/or synthetic origin, oligosaccharides of natural and/or synthetic origin, cyclodextrins of natural and/or synthetic origin, hyaluronates of natural and/or synthetic origin, carrageenans of natural and/or synthetic origin, derivatives thereof and mixtures thereof.
